(19) United States
(12) Patent Application Publication
(10) Pub. No.: US 2005/0250158 A1
(43) Pub. Date: Nov. 10, 2005

(54) Arrays of colloidal crystals

(76) Inventors: Atul Parikh, Woodland, CA (US); Adrian Brozell, Napa, CA (US); Michelle Muha, Novato, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 11/071,995

(22) Filed: Mar. 3, 2005

Related U.S. Application Data
(60) Provisional application No. 60/550,569, filed on Mar. 3, 2004.

(51) Int. Cl. G01N 33/53

(52) U.S. Cl. 435/7.1

(57) ABSTRACT

The present invention is directed to arrays of colloidal crystals and method of using such arrays to detect analytes in a sample.

### CROSS-REFERENCES TO RELATED APPLICATIONS

This application claims the benefit of U.S. Patent Application No. 60/550,569, filed Mar. 3, 2004, the disclosure of which is incorporated by reference in its entirety for all purposes.

### STATEMENT AS TO RIGHTS TO INVENTIONS MADE UNDER FEDERALLY SPONSORED RESEARCH OR DEVELOPMENT

This invention was made using government support under Grant No. PHY 0120999 from National Science Foundation and Grant No. DE-FG02-04ER46173 from the U.S. Department of Energy. The government has certain rights in this invention.

### BACKGROUND OF THE INVENTION

Organizing colloids of hundreds of nanometer to several micrometer in dimension are important in the design of optical and optoelectronic components (e.g., polarizers, filters, waveguides, and photonic circuits), nanoporous templates for applications in separation, filtration, and sensing, and as photonic transducers of chemical and biological signals.

The ability of monodisperse colloids to spontaneously organize into a face-centered cubic (f.c.c) lattice (Woodcock, L. V. Nature 385:141 (1997)) over macroscopic areas has important practical ramifications. First, it provides a simple self-assembly route for the design of three-dimensionally ordered structures which exhibit periodic spatial variation in refractive index with lattice constants of the order of the wavelength of light (Miguez, H. et al., Langmuir 13:6009 (1997); Joannopoulos, J. D. et al., Nature 386:143 (1997)). Such structures, also called synthetic opals, can be used directly—or as a template for—photonic band-gap (PBG) crystals to confine, manipulate, and guide the propagation of light (Yablonovitch, E. Physical Review Letters 58:2059 (1987); Colvin, V. L. Mrs Bulletin 26:637 (2001)). Moreover, these colloidal crystals provide useful templates for the design of nanoporous materials (Vlasov, Y. A. et al., Nature 414:289 (2001); Velev, O. D. et al., Current Opinion in Colloid & Interface Science 5:56 (2000)) and are potential candidates as optical transducers for chemical and biological sensors (Kulinowski, K. M. et al., Advanced Materials 12:833 (2000); Gates, B. et al., Chemistry of Materials 11:2827 (1999)).

Several methods including sedimentation (Jiang, P. et al., Journal of the American Chemical Society 121:11630 (1999)), electrophoretic deposition (Holtz, J. H. et al., Nature 389:829 (1997)), substrate drawing (Ballato, J. Journal of the Optical Society of America B-Optical Physics 17:219 (2000)), physical confinement (Sharma, A. C. et al., Journal of the American Chemical Society 126:2971 (2004); van Blaaderen, A. et al., Nature 385:321 (1997)), shearing (Holgado, M. et al., Langmuir 15:4701 (1999)), and spinning (Jiang, P. et al., Chemistry of Materials 11:2132 (1999); Park, S. H. et al., Advanced Materials 10:1028 (1998)) have proved successful in producing planar colloidal crystals. These methods rely on controlled gravitational settling or controlled solvent evaporation from starting colloidal sols. Recently, significant attention has been focused on patterning (Lu, Y. et al., Langmuir 17:6344 (2001); Amos, R. M. et al., Physical Review E 61:2929 (2000); Xia, D. Y. et al., Nano Letters 4:1295 (2004); Wang D. Y. et al., Advanced Materials 16:244 (2004); van Blaaderen, A. Mrs Bulletin 29:85 (2004); Yao, J. M. et al., Advanced Materials 16:81 (2004); Yin, Y. D. et al., Journal of the American Chemical Society 123:8718 (2001) colloidal crystals into complex geometries useful for photonic device integrations and the design of sensor microarrays. In this regard, the use of patterns of electric field and those of substrate topographies have become popular (Amos, R. M. et al., Physical Review E 61:2929 (2000); Xia, D. Y. et al., Nano Letters 4:1295 (2004)). More recently, a chemical templating method based on wettability contrast has also been reported (van Blaaderen, A. Mrs Bulletin 29:85 (2004); Yao, J. M. et al., Advanced Materials 16:81 (2004); Yin, Y. D. et al., Journal of the American Chemical Society 123:8718 (2001)). This method relies on the selective wetting of chemically structured surfaces during withdrawals from the colloidal sol to guide the crystal formation in well-defined regions of the substrate surface.

However, several limitations remain. The field-assisted methods employ conducting substrates (e.g., ITO) and cannot be easily generalized. The physical templating method, on the other hand, requires expensive photolithographically fabricated substrates for complex geometries. The chemical templating method circumvents these limitations, but depends on meniscus stability, colloidal sol concentration, withdrawal speeds, as well as feature dimensions and orientations. As a result, uniform crystal thickness on features of different dimensions on single substrates cannot be easily obtained (Yao, J. M. et al., Advanced Materials 16:81 (2004)). Furthermore, in all the methods above, the nucleation and growth occurs independently when used for designing discrete crystal islands. Thus, the elements of the resulting crystal arrays lack uniformity in crystal structural properties (e.g., orientation and/or thickness).

There is a need in the art for arrays of colloidal crystals that have uniform structural and optical properties. The present invention addresses these and other needs.

### BRIEF SUMMARY OF THE INVENTION

One embodiment of the invention provides an array of colloidal crystals on a solid support and having uniform structural and photonic properties. The colloidal crystals are spaced apart from each other. In another embodiment, the colloidal crystals are at least about 500 nm in size. In a further embodiment, the array has a pitch of about 1:1.

In other embodiments, the colloids are selected from the group consisting of: polymeric colloids, inorganic colloids, metal colloids, ceramic colloids, coated colloids, semiconductor colloids, and combinations thereof. In another embodiment, the colloids are polystyrene colloids. In still another embodiment, the colloids are silica colloids. In yet another embodiment, the colloidal crystals comprise colloids from about 100 nm to about 10 μm in size.

In a further embodiment, the colloids are functionalized with a functional group selected from the group consisting of: a carboxyl, an amino, an amido, an amidino, and combinations thereof. In another embodiment, the colloids are functionalized with a lipid bilayer. In yet another embodiment, the colloidal crystals further comprise a capture reagent.

Another embodiment of the invention provides a method of preparing such arrays. The method involves first contacting the colloids with a chemical template having lyophilic and lyophobic regions. The colloids are then crystallized into the colloidal crystals. Finally, the chemical template is removed to prepare the array of colloidal crystals. In some embodiments, the method further comprises the step of physically confining said colloids prior to the contacting step.

In another embodiment, the colloids are at a concentration of about 20% to about 75% by volume prior to the contacting step. In other embodiments, the colloids are at a concentration of about 44% to about 56% by volume.

In other embodiments, the colloids are selected from the group consisting of: polymeric colloids, inorganic colloids, metal colloids, ceramic colloids, coated colloids, semiconductor colloids, and combinations thereof. In another embodiment, the colloids are polystyrene colloids. In still another embodiment, the colloids are silica colloids. In yet another embodiment, the colloidal crystals comprise colloids from about 100 nm to about 10 μm in size.

In a further embodiment, the colloidal crystals comprise colloids that are functionalized. In another embodiment, the colloids are functionalized with a functional group selected from the group consisting of a carboxyl, an amino, an amido and an amidino. In still another embodiment, the colloidal crystals further comprise a capture reagent. In yet another embodiment, the capture reagent is selected from the group consisting of: a receptor, a ligand, an antibody, a nucleic acid, a polysaccharide, and combinations thereof. In still yet another embodiment, the capture reagent is an antibody. In other embodiments, the colloids are functionalized with a lipid bilayer. In still other embodiments, each of said colloidal crystals of said array are functionalized with a lipid bilayer.

Another embodiment of the invention provides methods of detecting analytes in a sample using such arrays. The first step of the method involves contacting a sample suspected of containing the analyte with an array of colloidal crystals comprising colloidal crystals having uniform structural and photonic properties. The second step involves detecting binding of the analyte to the colloidal crystals.

In some embodiments, the sample is a biological sample. In another embodiment, the analyte is selected from the group consisting of: a polypeptide, a nucleic acid, a lipid, a polysaccharide, a bacteria, a virus, a trace-metal, and combinations thereof. In a further embodiment, the colloidal crystals comprise functionalized colloids.

In other embodiments, the detecting comprises measuring a change in a stop band property of the colloidal crystals. In still other embodiments, the stop band property is selected from the group consisting of: an intensity shift, a wavelength shift, a width shift, and combinations thereof. In a further embodiment, the detecting comprises spectroscopy.

In further embodiments, the invention provides an apparatus comprising an array of colloid crystals, a radiation source (e.g., UV, infared, or visible light) for directing radiation to the colloidal crystals; and a detector adapted to detect radiation from the colloidal crystals.

### BRIEF DESCRIPTION OF THE DRAWINGS

FIG. 1 is a graphic illustration depicting preparation of an array of colloidal crystals of the invention.

FIG. 2 are images of arrays of colloidal crystals and colloidal crystals of the invention. FIG. 2A is an optical image of an array of polystyrene colloidal crystals. FIG. 2B-C are optical images individual polystyrene colloidal crystals. FIG. 2D-E are SEM images of individual polystyrene colloidal crystals.

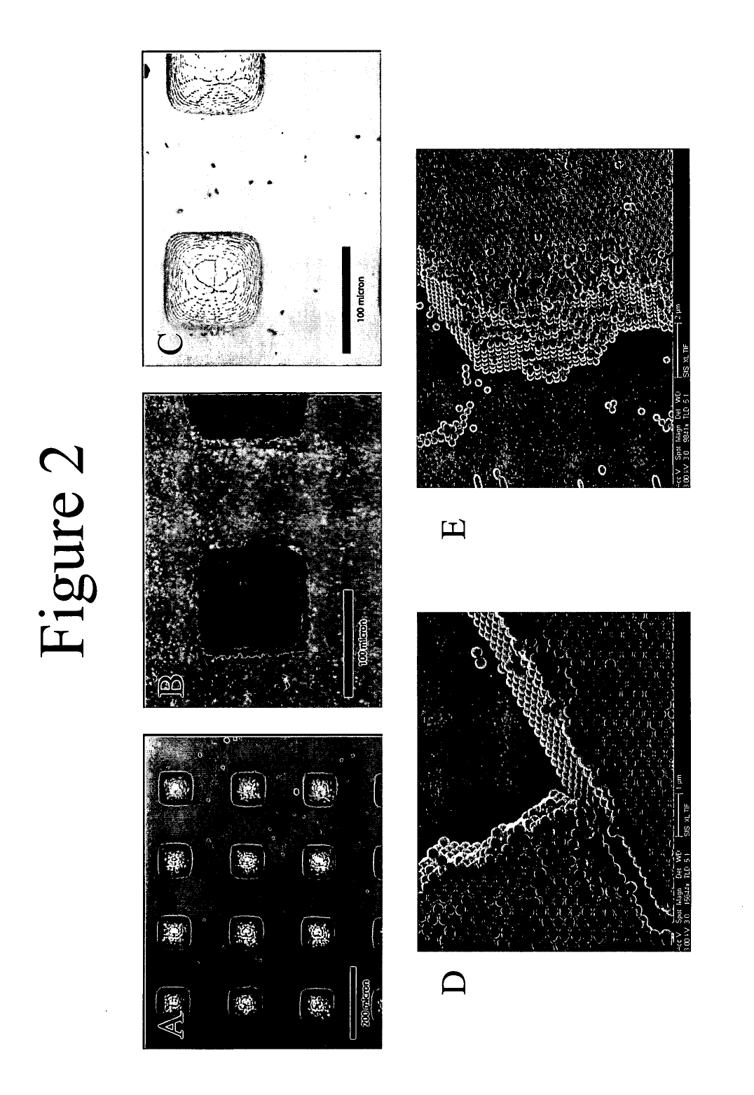

FIG. 3 depicts data demonstrating a change in the reflectance of a 330 nm silica colloidal crystal in water following addition of phosphate buffered saline.

FIG. 4 depicts data demonstrating a change in the transmittance of a 330 nm silica colloidal crystal in water following addition of phosphate buffered saline.

FIG. 5 depicts data demonstrating a change in the reflectance of a colloidal crystal comprising carboxyl-functionalized 250 nm polystyrene colloids and covalently linked to a goat anti-bovine antibody following contact with a mouse anti-goat antibody.

FIG. 6 depicts data demonstrating a change in the transmittance of a colloidal crystal comprising carboxyl-functionalized 250 nm polystyrene colloids and covalently linked to a goat anti-bovine antibody following contact with a mouse anti-goat antibody.

FIG. 7 depicts data demonstrating that there is no change in the reflectance of a colloidal crystal comprising carboxyl-functionalized polystyrene colloids and covalently linked to a goat anti-bovine antibody following addition of phosphate buffered saline.

FIG. 8 depicts data demonstrating the band gap shift that occurs as colloidal crystals are formed. FIG. 8A depicts the band gap shift for 240 nm polystyrene crystals as they dry at room temperature. FIG. 8B depicts the band gap shift for 330 nm silica crystals as they dry at 40° C.

FIG. 9 is a graphic illustration depicting the use of an array of colloidal crystals to translate a biological binding signal into an amplified optical read-out.

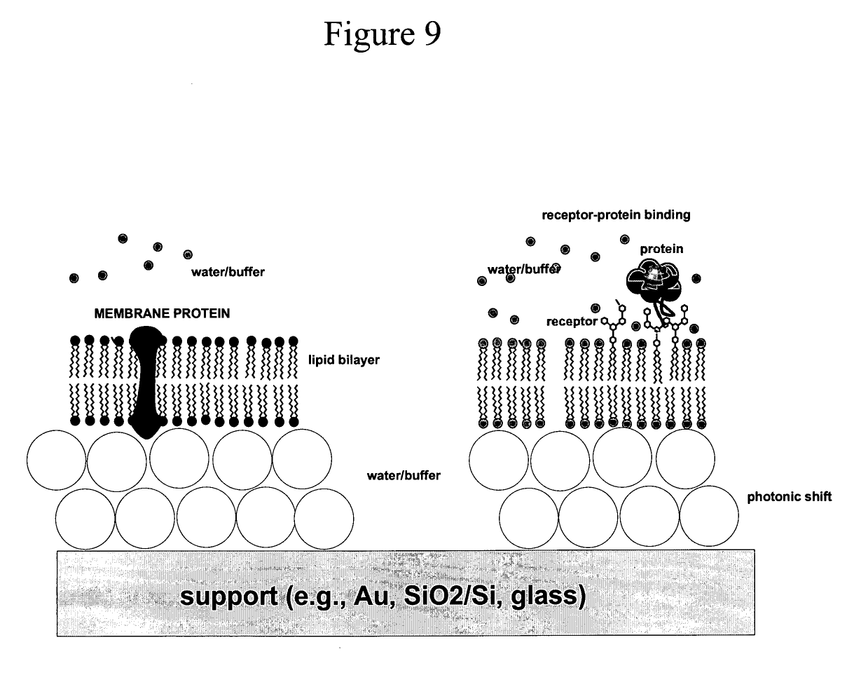

FIG. 10 depicts data demonstrating detection of the photonic stop band of an array of 330 nm silica crystals functionalized with a continuous fluid lipid bilayer.

### DESCRIPTION OF THE INVENTION

I. Introduction

The present invention is based on the surprising discovery that physical confinement employed in conjunction with a substrate surface displaying pre-patterned variations of interfacial energies results in the formation of well-defined arrays of three-dimensional colloidal crystals having uniform optical and structural properties. The colloids order into an f.c.c. lattice with their (111) planes perpendicular to the substrate surface and the arrays “epitaxially” reflect the underlying pattern of the substrate hydrophilicity. This hierarchical order was achieved by a slow evaporation of solvent from a concentrated aqueous colloidal sol sandwiched between a clean, hydrophilic glass and a patterned wettability glass surface. The disassembly of the sandwich resulted in two complementary micrometer scale spatial patterns of colloidal crystals which reflect the pattern of substrate hydrophilicity. Without being bound by theory, it is postulated that the capillary forces order the colloids into a large crystal within the sandwich (primary self-assembly) which subsequently cleaves with a remarkable regularity at the hydrophilic/hydrophobic boundary of the patterned substrates (secondary patterning). Because are arrays were generated from a single parent crystal, each element (i.e., each colloidal crystal in the array) is structurally (e.g., orientationally and morphologically) similar to every other element in the array. Furthermore, the technique was found applicable for larger micrometer-scale colloids as well as for variously functionalized colloids. We demonstrated the approach using monodisperse colloids of silica (330 nm and 5.66 μm) and polystyrene (PS, 240 nm, 260 nm, and 5.43 μm) and carboxylated PS (250 nm). High-density arrays of colloidal crystals with features as small as 100 μm² separated by distances as small as 100 μm could be routinely obtained, and the crystal thickness could be conveniently controlled by inserting a spacer between the substrates or varying the amount and/or concentration of the colloidal sol.

Our results suggest a general methodology for the construction of two-dimensional patterns of three-dimensionally ordered photonic colloidal crystals using a simple, inexpensive, and versatile means applicable to a wide-range of bead-types, dimensions, and surface functionalities.

Patterning of colloidal crystals through chemical patterning and physical confinement provides useful insights into the process of self assembly. Previous reported techniques by Fustin et al., Adv. Mat. 15(12) (2003), used chemically patterned substrates showing wettability contrast in conjunction with the substrate withdrawal from the colloidal solution. Their approach also results in the formation of colloidal arrays but the structures, thicknesses, and morphologies vary depending on the feature sizes of the initial chemical template. Further the edges of the features in their compositions reveal a thickness gradient and different structural order. As a result, crystal arrays of uniform structural and morphological properties cannot be obtained by their method.

A useful feature of our strategy is that larger size beads, from about 800 nm to about 5 μm, can also be crystallized into colloidal crystals. The competing gravitational effects play a smaller role due to the physical confinement of the colloidals prior to crystallization. The evaporation and withdrawal methods currently used in the art are affected by when using larger beads since the larger beads do not remain in the solution long enough to be deposited on to the substrate. Since the colloidal crystals prepared using the methods of the invention are crystallized through physical confinement, competing influences from gravitational sedimentation are absent.

The arrays of colloidal crystals described herein can conveniently be used for any detection method involving an optical or structural signal.

II. Definitions

As used herein, the term “array of colloidal crystals” refers to an organized arrangement of individual colloidal crystals that are comprised of colloids that have been crystallized or co-crystallized. The colloidal crystals are on a solid support and are spaced apart from each other.

A “structural property” as used herein refers to a physical property of an individual crystal such as size, shape, density, thickness, packing arrangement, orientation and morphology.

A “photonic property” or “optical property” as used herein refers to physical characteristics demonstrated when a colloidal crystal interacts with lightwaves and include, e.g., absorption, refraction, reflection, or transmittance of light waves. Photonic or optical properties include, for example, color, absorption, fluorescence, scattering, luminescence, brightness, transmittance or reflectance. “Absorption” or “absorptivity” refers to the fraction of light waves that are absorbed by a crystal. “Reflectance” or “reflectivity” refers to the fraction of the total radiant flux incident upon a surface (i.e., the surface of a colloidal crystal) that is reflected. Reflectance varies depending on the wavelength distribution of the incident radiation following contact between the light waves and the colloidal crystal. “Transmittance” refers to the fraction of light waves that reaches the boundary of the colloidal crystal. The term photonic also encompasses any wavelengths of light that are diffracted by the crystal.

As used herein, the term “pitch” refers to the spacing of features (e.g., colloidal crystals) in reference to the size of the features. A pitch of 1:1 means that the spacing between the features is equal to the size of the features;a pitch of 2:1 means that the spacing between the features is twice the size of the features; a pitch of 3:1 means that the spacing between the features is three times the size of the features; pitch of 4:1 means that the spacing between the features is four times the size of the features, etc.

As used herein, the term “chemical template” refers to a substrate (e.g., a planar solid support) used to prepare a colloidal crystal array. The chemical template may be unpatterned or may be patterned (i.e., comprise lyophilic and lyophobic regions on a single template).

As used herein, the term “lyophilic” refers to the affinity one material has for another material. Materials that are lyophilic have an affinity for each other and can coexist in close proximity. The term “lyophilic” includes the term “hydrophilic”, the affinity of a material for water.

As used herein, the term “lyophobic” refers to the repellant nature one material has for another material. Materials that are lyophobic repel one another and avoid contact with each other. The term “lyophobic” includes the term “hydrophobic”, the repellant nature of a material for water.

As used herein, the term “stop band” or “photonic gap band” refers to the range of wavelengths that are diffracted or reflected by the colloidal crystal. The stop band includes the “band-center” which refers to the wavelength that is most prominently diffracted by the crystal, and the “width of the stop band” which refers to the range of wavelength on either side of the band-center for which non-vanishing diffraction by the crystal occurs. The central wavelength of the stop band is proportional to the distance between each layer of beads in a colloidal crystal and is dependent on the index of refraction of the colloids. The spectrum of the stop band shows the reflectance of wavelengths in the stop band with the most light being reflected at the central wavelength.

“Sample” as used herein is an aqueous solution comprising an analyte of interest, i.e., any compound whose presence can be detected by detecting a change in the photonic stop band of a colloidal crystal following contact between the colloidal crystal and the compound. Analytes of interest include organic and inorganic substances and include, e.g., trace metals, polypeptides such as, immunoglobulins, ligands, counterligands, receptors; cofactors, toxins, enzymes (e.g., kinases, phosphatases, dehydrogenases, and the like), nucleic acid binding proteins (polymerases, histones, and the like); nucleic acids (e.g., genomic DNA, cDNA, RNA ssDNA, ssRNA, dsDNA, dsRNA, siRNA, mRNA, tRNA), glycoproteins, lipids (e.g., fatty acids such as myristic acid, palmitic acid, palmitoleic acid, stearic acid, oleic acid, linoleic acid, linolenic acid, and arachidonic acid; sterols such as cholesterol; and sphingolipids such as sphingomyelins and glycosphingolipids), and polysaccharides (e.g., carbohydrates, lectins, and the like). Samples include biological samples and chemical samples, waste-water samples, and other pools of aqueous reservoirs where analytes are likely to be present (e.g, stagnant water pools).

“Biological sample” as used herein is a sample of biological tissue or fluid that is suspected of containing an analyte of interest. Samples include, for example, body fluids such as whole blood, serum, plasma, cerebrospinal fluid, urine, lymph fluids, and various external secretions of the respiratory, intestinal and genitourinary tracts such as tears, saliva, semen, milk, and the like; and other biological fluids such as cell culture suspensions, cell extracts, cell culture supernatants. Samples may also include tissues biopsies, e.g., from the lung, liver, brain, eye, tongue, colon, kidney, muscle, heart, breast, skin, pancreas, uterus, cervix, prostate, salivary gland, and the like. A sample may be suspended or dissolved in, e.g., buffers, extractants, solvents, and the like. A sample can be from any naturally occuring organism or a recombinant organism including, e.g., viruses, prokaryotes or eukaryotes, and mammals (e.g., rodents, felines, canines, and primates). The organism may be a nondiseased organism, an organism suspected of being diseased, or a diseased organism. A mammalian subject from whom a sample is taken may have, be suspected of having, or have a disease such as, for example, cancer, autoimmune disease, or cardiovascular disease, pulmonary disease, gastrointestinal disease, muscoskeletal disorders, central nervous system disorders, infectious disease (e.g., viral, fungal, or bacterial infection). The term biological sample also refers to research samples which have been deliberately created for the study of biological processes or discovery or screening of drug candidates. Such examples include, but are not limited to, aqueous samples that have been doped with bacteria, viruses, DNA, polypeptides, natural or recombinant proteins, metal ions, or drug candidates and their mixtures.

A “capture reagent” as used herein refers to a moiety that binds to an analyte of interest. In some cases, capture reagent is a binding partner for the analyte of interest. For example, if a capture tag comprises the ligand component of a ligand-receptor combination, the analyte comprises the receptor component of the ligand-receptor combination. Conversely, if a capture reagent comprises the receptor component of a ligand-receptor combination, the analyte comprises the ligand component of the ligand-receptor combination. Suitable capture reagents include, polypeptides (e.g., avidin, streptavidin, or antibodies), nucleic acids, lipids, and polysaccharides. Other examples of capture agents include chemical and pharmaceutically relevant capture reagents (e.g., cyclodextrin family of compounds).

III. Preparation of Arrays of Colloidal Crystals

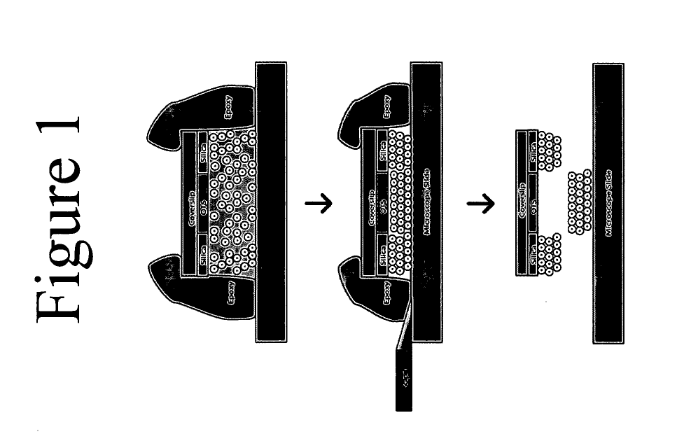

The arrays of colloidal crystals of the present invention are prepared by the steps of: (a) depositing colloids on a substrate; (b) contacting the colloids with a chemical template having lyophilic and lyophobic regions; (c) crystallizing the colloids into colloidal crystals; and (d) removing the chemical template, thereby preparing an array of colloidal crystals. An exemplary strategy used to form an array of colloidal crystals is shown in FIG. 1.

A. Contacting Colloids with a Chemical Template

Prior to contacting the colloids with a chemical template having lyophilic (e.g. hydrophilic) and lyophobic (e.g. hydrophobic) regions, the colloids to be used are prepared in a solution mixture and deposited onto a support substrate. The chemical template is then brought into contact with the solution of colloids on the support substrate and held in place while the colloids are crystallized. The chemical template has regions of hydrophobicity and regions of hydrophilicity that causes the solution of colloids in the hydrophilic region of the chemical template to interact with the chemical template. The solution of colloids in the hydrophobic region of the chemical template interacts with the chemical template to a much lesser degree. The solvent used to deposit the colloids on the support substrate evaporates, promoting the crystallization of the colloids into a colloidal crystal (see below). The positive interaction of the crystallizing colloids in the hydrophilic region of the chemical template is what allows selective removal of the colloids in the hydrophilic region upon removal of the chemical template. Furthermore, several orientations of the support substrate and chemical template are useful in the present invention. The support substrate and chemical template are typically in a parallel orientation with the support substrate on the bottom and the chemical template on top (see FIG. 1). Other useful orientations include those where the chemical template is on the bottom, or where the support substrate and chemical template are in a vertical orientation. As discussed in detail below, the contacting of the colloids with a chemical template is performed under conditions appropriate to promote the crystallization of the colloids into a colloidal crystal.

1. Substrates

The colloids are physically confined in an apparatus having an unpatterned substrate (support substrate) and a patterned substrate (i.e., a chemical template having lyophilic and lyophobic regions).

Each of the substrates used in preparation of an array of colloidal crystals of the present invention can be any metal oxide surface. Metals useful in the present invention include metals such as Si, Ti, Al, Ge, Au, Ag, Pd and Pt, as well as all other transition (Sc, V, Cr, Mn, Fe, Co, Ni, Cu, Zn, Y, Zr, Nb, Mo, Tc, Ru, Rh, Cd, La, Hf, Ta, W, Re, Os, Ir, Hg and Ac) and post-transition metals (Ga, In, Tl, Sn, Pb, Sb, Bi, and Po). Combinations of metals are also useful, and include, but are not limited to, GaAs. Oxidized organic materials such as oxidized polymeric surfaces can also be used in the preparation of an array of colloidal crystals of the present invention. Other materials useful as substrates include glass and alumina. One of skill in the art will appreciate that other materials are useful as substrates in the present invention.

The substrates useful in the present invention can be modified. One modification useful in the present invention involves the oxidation of a surface using a solution of hydrogen peroxide and concentrated sulfuric acid. This mixture oxidizes any material present on the surface, thereby removing any organic contaminants and oxidizing the substrate surface, for example, creating a metal oxide when the substrate is a metal.

Substrates useful in the present invention can also be patterned using traditional photolithographic methods as described in, e.g., Dulcey et al., Science 252:551 (1991) and Jonas et al., PNAS USA 99:5034 (2002), as well as the established methods of micro-contact printing as described in, e.g., Kumar et al., Langmuir 10:1498-1511 (1994). Use of photolithography can entail prior modification of the surface with a self-assembled monolayer, followed by exposure of the substrate surface through a mask such that the substrate surface in the exposed regions is further modified, and the unexposed regions of the substrate surface remain unchanged. Prior to the photolithography step, the substrate surface can be modified using self-assembled monolayers. The monolayers can be made of simple organic molecules, polymers, or biological materials such as proteins, nucleic acids and peptides. The self-assembled monolayers can be assembled using the procedures known in the art. For example, assembly of a small molecule having a tri-chlorosilane moiety can be accomplished by placing the substrate to be modified in a solution containing the small molecule and allowing the molecule to self-assemble onto the substrate surface. Additional methods of functionalization include vapor deposition.

Patterning via microcontact printing involves inking a stamp with the molecule to be assembled on the surface and then contacting the inked stamp with the substrate in order to transfer the molecule to the substrate surface.

The size of the patterned features is partly dependent on the method of pattern transfer used. Micro-contact printing can produce features that are limited in size by the stamp used to do the pattern transfer. Conventional lithography techniques using deep-UV exposure tools is limited by the wavelength of light used. Other techniques useful in the present invention include step-and-flash imprint lithography, electron-beam, scanning-tunneling microscopy and dip-pen nanolithography. One of skill in the art will appreciate that other methods of patterning are also useful in the present invention.

2. Colloids

Any suitable colloidal particles of any shape can be used. The particles are chosen depending upon the optimum degree of ordering and the resulting lattice spacing desired for the particular application. Colloids useful in the present invention can be made from inorganic substances such as silica and alumina, as well as metals such as transition metals, post-transition metals and semiconductors. Colloids useful in the present invention can also be made from polymeric materials such as styrenics (such as polystyrene), methacrylics (such as polymethylmethacrylate), acrylics and fluorinated polymers such as polytetrafluoroethylene. One of skill in the art will appreciate that additional polymeric materials are useful in making the colloids of the present invention. Other useful colloidal materials include ceramics, coated colloids and combinations of materials. The colloids useful in the present invention can be purchased from commercial vendors or prepared using techniques known to one of skill in the art.

The colloids of the present invention can comprise a single material, such as a silica colloid or a polystyrene colloid, or they can comprise a combination of materials. Colloids of the present can comprise a combination materials including a combination of metals, inorganic substances or polymeric materials. In addition, the colloids of the present invention can comprise a polymeric material in combination with a metal or an inorganic material. One of skill in the art will appreciate that other combinations of materials are useful in the colloids of the present invention.

When comprised of a single material, the colloids of the present invention are homogeneous. When comprised of a combination of materials, the colloids of the present invention can be a homogeneous mixture of the combination of materials, or the different materials can be separated into different regions of the colloids. For example, a colloid comprising a polymer and an inorganic material can have the inorganic material at the core and the polymeric material on the exterior of the colloid. One of skill in the art will appreciate that colloids having at least two layers of materials are useful in the present invention, and that the composition and thickness of each layer can be adjusted to meet the need of one of skill in the art.

The colloids of the present invention can also be functionalized. In some embodiments, the colloids are functionalized with groups such as carboxyl groups, amino groups, amido groups or amidino groups. In some embodiments, the functional groups include capture reagents (e.g., proteins, polypeptides, polysaccharides, bacteria, viruses or metals). Other capture reagents useful for functionalizing the colloids of the present invention include lipids and lipid bilayers. The lipids and lipid bilayers can be applied to the colloids prior to crystal formation, or after formation of the array of colloidal crystals. One of skill in the art will appreciate that other functional groups are useful for functionalizing the colloids of the present invention. Functionalization of the colloids can occur either prior to or after preparation of the array of colloidal crystals of the present invention. For other functional groups, one of skill in the art will appreciate that the appropriate reaction conditions for functionalizing the colloid can be dependent on the functional group being used.

Colloids useful in the present invention can be of any size on the nanometer to the micrometer scale. Colloids useful in the present invention include colloids with a size from about 1 nm to about 1 mm, about 10 nm to about 100 μm, about 50 nm to about 700 nm, about 100 nm to about 10 μm, about 200 nm to about 500 nm, about 400 nm to about 700 nm, about 300 nm to about 1 μm, about 500 nm to about 2 μm, about 750 nm to about 2 μm, or about 5 nm to about 6 μm. One of skill in the art will appreciate that colloids of other sizes are also useful in the present invention.

Solvents useful for preparing mixtures of colloids of the present invention include, but are not limited to, water, alcohols (such as ethanol and propanol) and any polar, protic solvent. Solutions of colloids useful in the present invention can have concentrations from 20% to about 75% , 30% to about 70%, 40% to about 60%, or about 44% to about 56% by volume. One of skill in the art will appreciate that other concentrations are useful in the present invention.

B. Crystallization of Colloids into Colloidal Crystals

Crystallization of the colloids into colloidal crystals is accomplished by promoting the evaporation of the solvent used to deposit the colloids onto the support substrate. The conditions used for the crystallization step can be dependent on the solvent used, the type and size of the crystal used, the concentration of the colloid solution deposited onto the support substrate, the temperature during crystallization, as well as other factors apparent to one of skill in the art. The use of a higher temperature can result in a shorter time for crystallization. In some embodiments, useful temperatures include those from about 5° C. to about 100° C., about 10° C. to about 80° C., about 20° C. to about 60° C., about 25° C. to about 50° C., about 30° C. to about 45° C., o about 35° C. to about 40° C. Useful times for crystallization include about 15, 30, or 45 minutes, 1, 2, 4, 6, 8, 10, 12, 26, 28, 20, 24, 48, 72, or 96, hours or about 5, 10, 15, or 20 days. Longer and shorter times for crystallization can also be useful in the present invention. The relative humidity of the atmosphere in which the crystallization is performed can be from about 10% to about 95%, about 20% to about 85%, about 30% to about 75% , about 40% to about 65%, or about 40% to about 55%.

Solvents useful in the crystallization of the colloidal crystals include, but are not limited to, water, alcohols (such as ethanol and propanol) and any polar, protic solvent. Solutions of colloids usefuil in the present invention can have concentrations from about 5%, 10%, 15%, 20% , 25%, 30%, 35%, 40%, 45%, 50%, 55%, 60%, 65%, 70%, or 75% by volume. One of skill in the art will appreciate that other concentrations are useful in the present invention.

One of skill in the art will appreciate that other variables including pH, salt concentration of the solvent, pressure, and ambient conditions can be varied to generate arrays of colloidal crystals having desired characteristics.

The crystallization of the colloids in this manner results in colloidal crystals having uniform structural and photonic properties. The crystal structure of the colloidal crystals can be face-centered cubic (f.c.c.) or body-centered cubic (b.c.c.) depending on the type and size of the colloid used, as well as the time, temperature and solvent used dunmg crystallization. Other crystal structures are also usefuil in the present invention.

C. Arrays

Removal of the chemical template from the support substrate results in preparation of the array of colloidal crystals of the present invention by removing the colloidal crystals that were in contact with the hydrophilic regions of the chemical template. The size, shape and pitch of the colloidal crystals are determined by the mask or stamp used in the patterning step. The colloidal crystals of the present invention can be of any size from about 500 nm to about 1 cm. The shape of the colloidal crystals of the present invention can be square, round, elliptical, triangular, rectangular, rhombal and toroidal. Other shapes are also useful in the present invention. The pitch of the array of colloidal crystals can be from about 1:1 (space between crystals:size of crystal) to about 10:1, about 9:1, about 8:1, about 7:1, about 6:1, about 5:1, about 4:1, about 3:1, about 2:1, or about 1:1. Other pitches are also useful in the present invention.

The colloidal crystals of the present invention also have useful structural and photonic properties. Useful structural properties include, but are not limited to, swelling and contracting of the colloidal crystals following a binding event. Useful photonic properties include, but are not limited to, as dispersion, reflection and stop band properties. Other photonic and structural properties of the colloidal crystals are useful in the present invention.

The arrays of colloidal crystals of the present invention can be further derivatized with small molecules or biological materials. In some embodiments, the colloids are functionalized with groups such as carboxyl groups, amino groups, amido groups or amidino groups. One of skill in the art will appreciate that other functional groups are useful for functionalizing the colloids of the present invention. In other embodiments, each colloidal crystal member of the array can be functionalized with a capture reagent (e.g., proteins, polypeptides, lipids, polysaccharides, bacteria, viruses or metals). The colloidal crystals of the array can all have the same capture reagent or each can have a different capture reagent. Binding of an analyte of interest to a capture reagent can be detected by detecting shifts in the optical or structural of the colloidal crystals using the methods described in detail below.

In still other embodiments, the array of colloidal crystals is functionalized with a lipid bilayer. The lipid bilayer on each crystal on an array of colloidal crystals can be the same or different. In some embodiments, the entire array of colloidal crystal is functionalized with a single lipid bilayer. The lipid bilayer may be formed from phospholipid based multilamellar vesicles (MLVs) and small unilamellar vesicles (SUVs). MLVs and SUVs are concentric bilayer vesicles containing an aqueous solution in the core and typically have diameters of from about 25 nm to 4 m and from about 200 to about 500 Å, respectively. Methods of generating MLVs and SUVs are well known in the art and are set forth in Example 6 below.

In some embodiments, colloidal crystals functionalized with continuous lipid bilayers are used to study interactions between different types of biological molecules, e.g., transmembrane proteins and their ligands or cell surface receptors and their ligands. The bilayers prevent nonspecific interactions and allow detection of specific reactions between biological molecules. In some embodiments, the lipid bilayer functionalized arrays of colloidal crystals can be used to identify compounds (e.g., drugs, pathogens such as anthrax toxin, and polypeptides) that bind to or modulate the activity of transmembrane proteins or receptors. For example, binding of test compounds to the transmembrane protein or receptor can be detected by detecting changes in the optical or structural properties of the colloidal crystals. In some embodiments, each crystal on an array of colloidal crystals is functionalized with a lipid bilayers containing different transmembrane proteins or receptors, thereby allowing multiplex analysis of the effects of the same analyte on different transmembrane proteins or receptors. Methods for detecting analytes that bind to lipid bilayers containing different transmembrane proteins or receptors are set forth in U.S. patent Publication No. 20040180147.

IV. Detection of Analytes

In one embodiment of the invention, the arrays of colloidal crystals can conveniently be used to detect analytes in a sample. Detection of analytes is based on the photonic properties or the structural properties of the colloidal crystals. Interaction with an analyte of interest induces a change in the photonic or structural property of the colloidal crystal which can be detected using any means known in the art. For example, the stop band properties, the dispersion properties or changes in the shape of the colloidal crystals of the invention can be used to detect binding of analytes to the colloidal crystals.

In some embodiments, the stop band or gap band properties of the colloidal crystals of the invention are used to detect binding of an analyte of interest to the crystal. The stop band and changes in the stop band following binding of an analyte of interest to the crystal can be detected by, e.g., measuring reflected light or transmitted light. Measure of the change in transmission or reflection intensity of light at any of stop band wavelengths. One of skill in the art will appreciate that colloidal crystal comprising different types of materials will have different stop bands. Typically a stop band will be ˜50 nm for polystyrene and silica. One of skill in the art will appreciate that any method that measures stop bands could be used to measure analyte binding. Typically, binding of an analyte of interest to a colloidal crystal will induce a shift in the stop band or stop band peak of at least about 1, 2, 4, 6, 8, 10, 20, 30, 40, 50, 60, 70, 80, 90, 100, 110, 120, 130, 140, 150, 160, 170, 180, 190, 200, 210, 220,230, 240, 250, 260, 270, 280, 290, 300 nm higher or lower compared to the stop band or stop band peak in the absence of the analyte of interest.

In some embodiments, the dispersion property of the colloidal crystals of the invention is used to detect binding of an analyte of interest to the crystal. “Dispersion” as used herein refers to the index of refraction dependence on wavelength. Once the analyte binds to the colloidal crystal and it undergoes a conformational change, the index of refraction for all wavelengths will change. This change can be detected using any means known in the art. For example, the change can be detected by exposing the crystal to a light source such as a laser at a preset angle of incidence before, during, and/or after contacting the crystal with a sample suspected of containing an analyte of interest. A change in the location of the crystal's index of refraction in location indicates analyte binding. One of skill in the art will appreciate that any method that measures refractive index could be used to measure analyte binding (see, e.g., Tarhan and Watson, Physical Review 54(11):7593) (1996) and Yablonovitch, Physical Review Lett. 58(20): 2059 (1987)). Typically, binding of an analyte of interest to a colloidal crystal will induce a shift in the refractive index of at least about 10, 20, 30, 40, 50, 60, 70, 80, 90% or more than in the absence of the analyte of interest.

Interferometeric methods could also be used to measure analyte binding to the arrays of colloidal crystals. Interferometeric methods detect binding of an analyte of interest to a colloidal crystal based on the structural properties of the crystal. Upon binding of the analyte of interest to the colloidal crystal, the colloidal crystal will swell or deflate. For example, by using a Michelson interferometer, the swelling of the crystal could be measured to measure analyte binding. Typically, binding of an analyte of interest to a colloidal crystal will induce swelling or deflation by at least about 10, 20, 30, 40, 50, 60, 70, 80, 90% or more than in the absence of the analyte of interest.

A. Devices for Analyte Detection

In embodiments of the invention, the array of colloidal crystals can be used with a radiation source and a detector to form an apparatus suitable for detecting binding events between an analyte in a sample and the colloidal crystal(s). The radiation source may be a light emitting source, which provides light having an intensity and wavelength sufficient to excite the colloidal crystals. Suitable radiation sources are known to those of ordinary skill in the art and are commercially available. The detector may be an optical detector. The optical detector can be adapted to detect light emitted (e.g., transmitted) from the colloidal crystals or to detect changes in the direction of the light emitted from the colloidal crystals. Suitable optical detectors are also commercially available and are known in the art. A computer may be coupled to the detector and can provide suitable information regarding which colloidal crystal(s) in the array are bound to the analyte.

In certain embodiments, the arrays of the invention can be integrated into devices for detecting analytes. The arrays can be integrated into any device that can detect changes in the optical properties of the colloidal crystals described herein. Suitable devices typically include a light source and a detector that detects changes in the optical properties of the colloidal crystals. Such devices include spectrophotomers (UV and visible), laser-based devices (e.g., solid state lasers, gas lasers, semiconductor lasers, and dye lasers), biosensing devices, microfluidics devices, and optical waveguides In some cases, the devices may detect changes in other properties of the colloidal crystals described herein. Such devices include, e.g., interferometers.

For detection of analytes using a spectrophotometer, a spectrum is scanned over a range of wavelength to record the shape of the band gap of a colloidal crystal or array of colloidal crystals by measuring the intensity of the light that is transmitted through the colloidal crystal. Typically, successive scans are run to measure the jitter in the crystal, i.e., variations in the location of the stop band due to the Brownian motion of the colloids in the crystal. Scans are typically run before, during, and after contacting a sample with an array of the invention. Shifts in band gap are detected to detect the presence of an analyte of interest in the sample. In some embodiments, wavelength at the half maxima of the band gap is determined and a kinetic scan is run at that wavelength to detect changes in intensity of the light transmitted through the colloidal crystal. Detection of changes in the transmitted intensity at half maxima the presence of an analyte of interest.

In some embodiments, the arrays of colloidal crystals described herein are integrated into laser-based devices are used to detect analytes. Detection of changes in the index of refraction through a sample when the sample of contacted with an array of the invention detects an analyte of interest in the sample.

In some embodiments, the arrays of colloidal crystals described herein are integrated into optical waveguides. When there is a shift in the band gap of the crystals, light propagating through the guide will exit and there will be a measurable decrease in the intensity of light at the end of the guide.

B. High Through Put Screening

In certain embodiments, the arrays of the invention can be used in high throughput screening (HTS) methods. High throughput assays for evaluating the presence, absence, quantification, or other properties of particular nucleic acids, polypeptides, or chemical compounds are well known to those of skill in the art. Similarly, binding assays and reporter gene assays are similarly well known. Thus, e.g., U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate procedures, including sample and reagent pipeting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems.

Conventionally, new chemical entities with useful properties are generated by identifying a chemical compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

In one embodiment, high throughput screening methods involve providing a library containing a large number of potential therapeutic compounds (candidate compounds). Such “combinatorial chemical libraries” are then screened in one or more assays to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library, such as a polypeptide (e.g., mutein) library, is formed by combining a set of chemical building blocks called amino acids in every possible way for a given compound length (i.e., the number of amino acids in a polypeptide compound). Millions of chemical compounds can be synthesized through such combinatorial mixing of chemical building blocks (Gallop et al., J. Med. Chem. 37(9):1233-1251 (1994)).

Preparation and screening of combinatorial chemical libraries is well known to those of skill in the art. Such combinatorial chemical libraries include, but are not limited to, peptide libraries (see, e.g., U.S. Pat. No. 5,010,175, Furka, Pept. Prot. Res. 37:487-493 (1991), Houghton et al., Nature, 354:84-88 (1991)), peptoids (PCT Publication No WO 91/19735), encoded peptides (PCT Publication WO 93/20242), random bio-oligomers (PCT Publication WO 92/00091), benzodiazepines (U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with a Beta-D-Glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho, et al., Science 261:1303 (1993)), and/or peptidyl phosphonates (Campbell et al., J. Org. Chem. 59:658 (1994)). See, generally, Gordon et al., J. Med. Chem. 37:1385 (1994), carbohydrate libraries (see, e.g., Liang et al., Science 274:1520-1522 (1996), and U.S. Pat. No. 5,593,853), and small organic molecule libraries (see, e.g., benzodiazepines, Baum, C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, compounds that regulate adenyl cyclase and cyclic AMP, such as, for example, forskolin and its derivatives, U.S. Pat. Nos. 5,789,439; 5,350,864, and 4,954,642.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.), which mimic the manual synthetic operations performed by a chemist. The above devices, with appropriate modification, are suitable for use with the present invention. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

### EXAMPLES

### Example 1

### Preparation of Patterned and Unpatterned Substrate

Substrate preparation: The substrates used were 18 mm×18 mm coverslips (Corning no. 2) and 75 mm×25 mm pre cleaned microscope slides from Gold Seal. The substrates were cleaned from adventitious contaminants (Fan et al., Langmuir 20:3062 (2004)) by oxidizing in a freshly prepared “piranha-etch” solution comprising a 4:1 (v/v) mixture of sulfuric acid and hydrogen peroxide for a period of 4-5 minutes maintained at ˜100° C. The substrates were then withdrawn using teflon tweezers, rinsed immediately using deionized H₂O, and dried in a stream of nitrogen. All cleaned, oxidized substrates were used within 1 day of the pretreatment.

Surface modification with n-octadecyltrichlorosilane: All freshly oxidized, dry substrates were immersed in a 50 ml self-assembly solution consisting of 2.5 mM octadecyltrichlorosilane (CH₃(CH₂)₁₇SiCl₃, OTS) (90% Aldrich, Parikh et al., J. Phys. Chem. 98:7577-7590 (1994)) solution in anhydrous hexadecane (99% Sigma-Aldrich) (Hayward et al., Nature 404:56 (2000)). The substrates were allowed to incubate with the self-assembly solution for approximately 45 min. All silanization reactions were carried out in glass containers under nominally dry ambient conditions (relative humidity<20%). All the reaction vessels used were thoroughly pre-cleaned using Alconox detergent followed by extensive washing with deionized, low organic water and finally air-dried in an oven. After removal from the self-assembly solution, the film-covered substrates were washed extensively with chloroform under ultrasonic conditions to remove any excess reactants. Silanized samples thus obtained were used within a few days of preparation. Substrate silanization was confirmed by measuring high contact angle of water (>100 degrees) following the treatment.

Photolithographic patterning of surface modified with n-octadecyltrichlorosilane: Spatial patterning of OTS covered substrates was achieved using short-wavelength UV radiation (Brzoska et al., Nature 360:719 (1992); Parikh et al., Journal of Physical Chemistry 98:7577 (1994)). In particular, spatially-directed photoillumination of monolayer samples was achieved using a physical mask and an ozone generating UV lamp (Dulcey et al., Science, 252:551 (1991)). The masks displaying patterns of chrome over quartz substrate were either acquired from Photoscience, Inc (Torrance, Calif.) or produced at the UC Davis Microfabrication Facility (Lopez et al., Science 260:647 (1993). Masks acquired from Photoscience, Inc. contained square features ranging in size from 500 μm to 100 ηm and 1 mm to 5 μm, or were prepared at the UC Davis Northern California Nanotechnology Center.

UV radiation was produced using a medium-pressure Hg-discharge grid lamp (UVP, Inc., Upland, Calif.) in a quartz envelope, and maintained in a closed chamber in a chemical hood. The samples were placed in contact with the photomask and positioned approximately 0.5-2 mm from the light source depending on the illumination geometry. The exposure period was approximately 40-60 min depending on the exposure geometry (sample-lamp distance) and the age of the lamp.

Following the exposure, the mask was separated from the substrate surface, samples rinsed thoroughly using water, chloroform, and ethanol, and dried with nitrogen. Patterned OTS samples were used within 24 h of preparation.

### Example 2

### Preparation of an Array of Colloidal Crystals using Polystyrene Colloids

The 240 nm and 260 nm polystyrene colloids were purchased from Duke Scientific and the 5.43 μm polystyrene colloids were purchased from Bangs Laboratories. Highly concentrated sols in water were prepared by concentrating the solutions by centrifugation using a Fischer A Microcentrifuge. The polystyrene colloids were spun for 15 minutes at 9500 rpm. The supernatant, water, was removed to bring the concentration of colloids by volume to between 44% -56% . High concentration solutions were sonicated for approximately 5 minutes, then vortexed to resuspend the colloids.

Freshly concentrated colloids were sealed between recently oxidized, clean microscope slides and patterned OTS coverslips (prepared as described in Example 1) using Devon 2 Ton Epoxy. Samples were then left at room temperature for up to two weeks, or in an oven at 40° C. for −3 days. Lower concentration samples took longer to crystallize. Samples were opened by gently sliding a scalpel at the interface between the epoxy and the microscope slide.

Color images were captured with a Sony Exwave II camera connected to an optical microscope (Bruker Instruments, Mass.) to obtain optical images. After sputtering a 30 Å of gold on each sample, SEM images were produced with a FEI XL30-SFEG microscope. UV-Vis spectrums were recorded by a Cary 1e connected to a Pentium 133 MHz PC. Room temperature conditions were optimal for the preparing colloidal crystals from polystyrene beads.

Optical photographs reveal a representative crystal morphology obtained before the sample sandwich was disassembled. The images reveals a series of parallel stripes ˜300 um in width. Such stripes, reminiscent of stick-slip motion, have been observed (Dulcey et al., Science 252:551 (1991); Lopez et al., Science 260:647 (1993); Paulin et al., Physical Review Letters 64:2663 (1990)) previously in capillary forces driven assemblies of colloidal particles. They are generally attributed to the competition between the surface tension of the wetting film and the frictional force experienced by the contact line due to the convective transport of the colloidal particles to the evaporation boundary. Each stripe further reveals a pattern of hexagonal and parallel cracks between ˜100 um single crystal domains, also consistent with those observed previously. The crystalline order appear preserved across the cracks and the boundaries mirror each other, confirming that the polycrystallinity observed is not the result of uncorrelated nucleation processes, but form post-growth. The image for sub-micrometer colloids also reveals the presence of a faint, but reproducible outline reflecting the hydrophilic/hydrophobic edge of the OTS pattern. The outline further separates the brighter green from the fainter green color of the crystal and is most probably due to the dewetting of the crystallizing sol from the hydrophobic OTS parts of the sample sandwich resulting in slightly different crystal thicknesses on the hydrophilic and the hydrophobic parts of the substrate. Because this height difference is expected to be small, we do not observe the outline for micrometer scale beads.

When the sandwich cell was disassembled, the colloidal crystal cleaved with a remarkable reproducibility along the hydrophilic/hydrophobic boundary. The colloidal phase was retained on the hydrophilic regions of the patterned OTS surface and the complementary crystal phase was observed for the uniformly hydrophilic silica substrate. These show that the entire crystal is preserved on one of the two bounding surfaces. The cleavage occurs preferentially at the substrate planes rather than at other arbitrary planes within the crystal on several parts of the substrate. Occasionally, a partial cleavage leaving behind residual crystal on each of the two bounding surfaces was also observed. The FE-SEM images further show that the layers retain their essential f.c.c. crystallographic ordering on each of the two surfaces and across the crystal cracks. Because the cleaved crystals were generated from the original master single crystal (albeit cracked), the elements of the array preserve the ordering, thereby forming an array of microscopic colloidal crystals in single, uniform orientation.

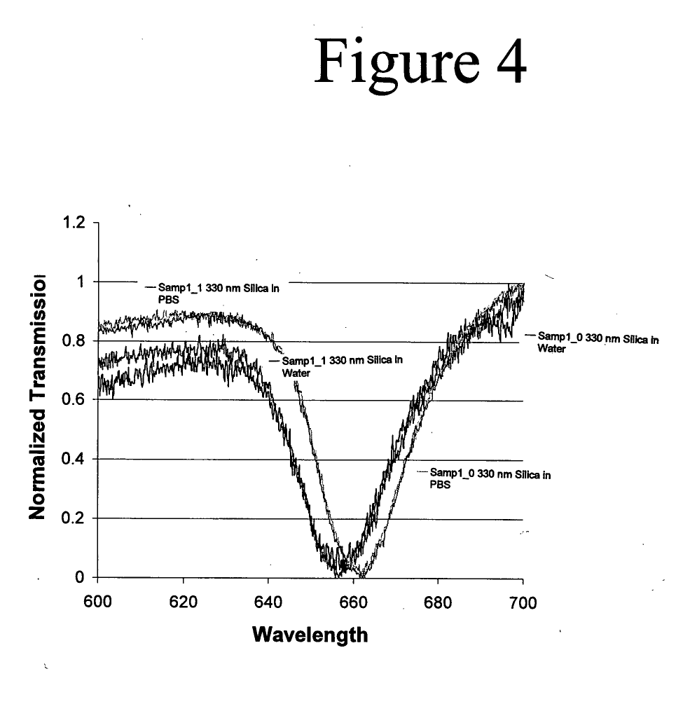

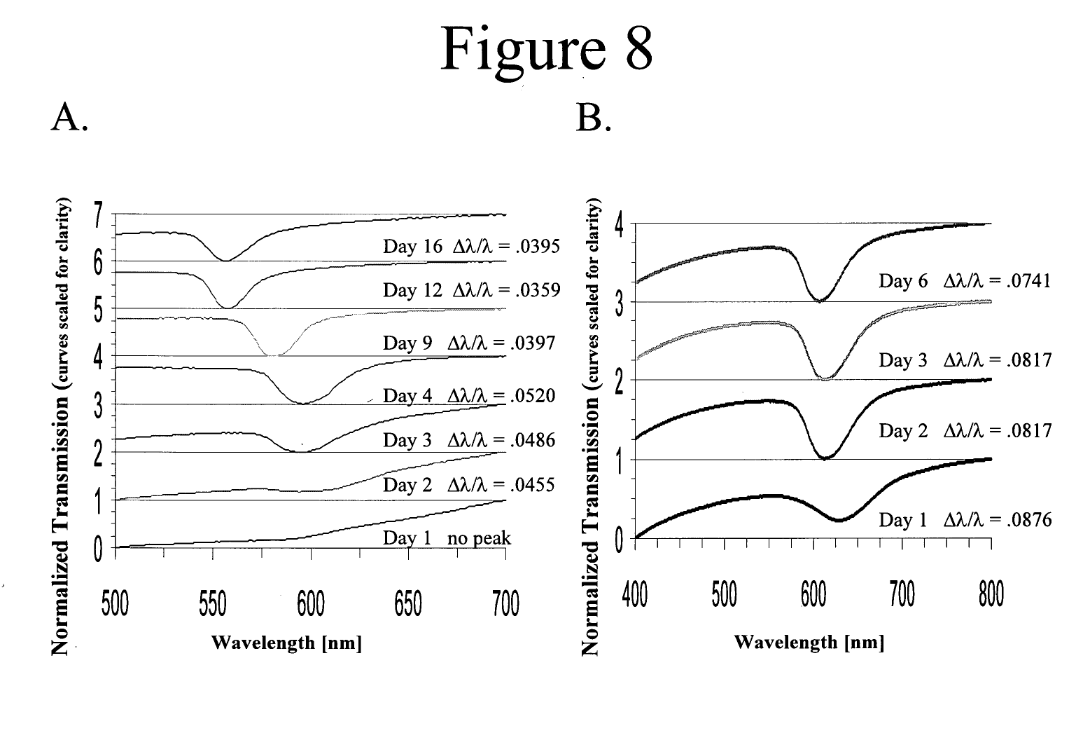

These findings are further confirmed by the optical properties of these crystals. Normal incidence transmission spectra shown in FIG. 4 reveal a characteristic dip in the transmission traces which correspond well with the expected stop-band for the colloidal dimensions. The observation of the changes in the spectral properties during the drying period sheds light on the ordering process. In all cases, the beads ordered rapidly as indicated by the early appearance of the stop-band peak. Over time, a slight blue shift (decrease in the photonic stop-band wavelength) was observed, which is consistent with a tighter packing achieved as a result of drying. Final peak positions observed for 240 nm and 260 nm polystyrene were 530 nm, and 580 nm, respectively. The slightly lower than theoretical values obtained for stop band peak positions is consistent with previous studies employing aqueous phase assembly. In samples that were dried at room temperature, peak location shifted ˜20 nms when the final traces of water were removed by opening the confinement cell. The final location was similar to samples dried at elevated temperatures (40° C.). This data is shown in FIG. 8.

### Example 3

### Preparation of an Array of Colloidal Crystals using Silica Colloids

The 330 nm and 5.66 micron silica colloids were purchased from Bangs Laboratories. Highly concentrated sols in water were prepared by concentrating the solutions by centrifugation using a Fischer A Microcentrifuge. The polystyrene colloids were spun for 15 minutes at 9500 rpm. The supernatant, water, was removed to bring the concentration of colloids by volume to between 44% -56% . High concentration solutions were sonicated for approximately 5 minutes, then vortexed to resuspend the colloids.

Freshly concentrated colloids were sealed between recently oxidized, clean microscope slides and patterned OTS coverslips (prepared as described in Example 1) using Devon 2 Ton Epoxy. Samples were then left at room temperature for up to two weeks, or in an oven at 40° C. for ˜3 days. Lower concentration samples took longer to crystallize. Samples were opened by gently sliding a scalpel at the interface between the epoxy and the microscope slide.

Color images were captured with a Sony Exwave II camera connected to an optical microscope (Bruker Instruments, Mass.) to obtain optical images. After sputtering a 30 Å of gold on each sample, SEM images were produced with a FEI XL30-SFEG microscope. UV-Vis spectrums were recorded by a Cary 1e connected to a Pentium 133 MHz PC. The highest quality silica crystal arrays were achieved at 40° C.

When the sandwich cell was disassembled, the colloidal crystal cleaved with a remarkable reproducibility along the hydrophilic/hydrophobic boundary. The colloidal phase was retained on the hydrophilic regions of the patterned OTS surface and the complementary crystal phase was observed for the uniformly hydrophilic silica substrate. These show that the entire crystal is preserved on one of the two bounding surfaces. The cleavage occurs preferentially at the substrate planes rather than at other arbitrary planes within the crystal on several parts of the substrate. Occasionally, a partial cleavage leaving behind residual crystal on each of the two bounding surfaces was also observed. The FE-SEM images further show that the layers retain their essential f.c.c. crystallographic ordering on each of the two surfaces and across the crystal cracks. Because the cleaved crystals were generated from the original master single crystal (albeit cracked), the elements of the array preserve the ordering, thereby forming an array of microscopic colloidal crystals in single, uniform orientation.

These findings are further confirmed by the optical properties of these crystals. Normal incidence transmission spectra shown in FIG. 4 reveal a characteristic dip in the transmission traces which correspond well with the expected stop-band for the colloidal dimensions. The observation of the changes in the spectral properties during the drying period sheds light on the ordering process. In all cases, the beads ordered rapidly as indicated by the early appearance of the stop-band peak. Over time, a slight, but measurable blue shift (decrease in the photonic stop-band wavelength) occurred which is consistent with tighter packing which is achieved as a result of drying. Final peak positions observed for 330 nm silica beads was approximately 600 nm. The slightly lower than theoretical values obtained for stop band peak positions is consistent with previous studies employing aqueous phase assemblyⁱⁱⁱ. In samples that were dried at room temperature, peak location shifted up to ˜30 nm when the final traces of water were removed by opening the confinement cell. The final location was similar to samples dried at elevated temperatures (40° C.). This data is shown in FIG. 8.

### Example 4

### Changes in the Reflectivity of a Silica Crystal

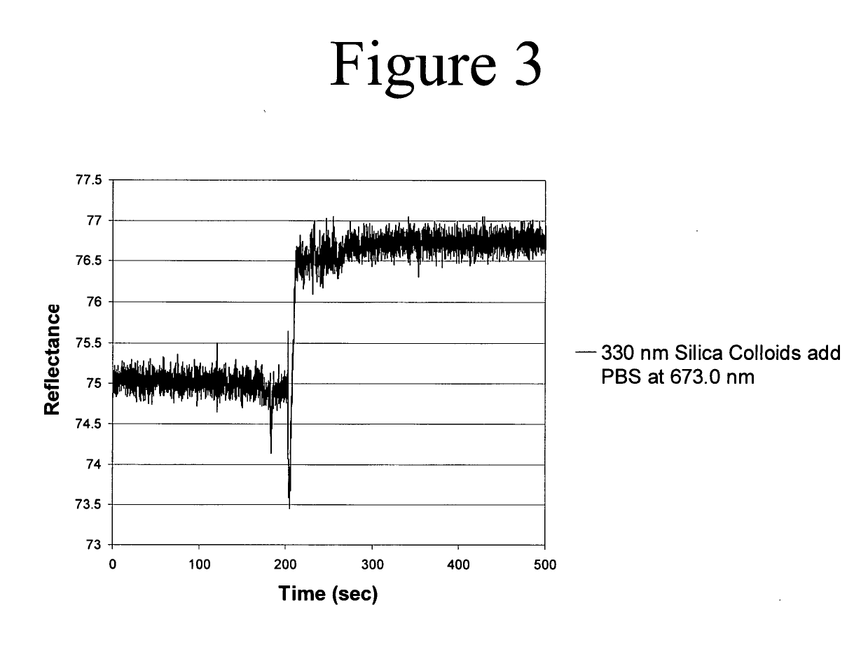

An array of 330 nm silica crystals was prepared as described in Example 3 above and was stored in H₂O. A continuous transmission intensity measurement was performed at 673 nm for ˜2 minutes to get a baseline reading before adding phosphate buffer saline (PBS) via syringe pump to the system. Following addition of PBS to the array, the reflectivity of the array measured at 673 nm dramatically increased from 75% to 76.5%. This data is illustrated in FIG. 3. Several ˜200 nm spectral scans, before and after the addition of PBS, illustrate the shift in the photonic stop band, FIG. 4.

### Example 5

### Detection of a Target Polypeptide using an Array of Functionalized Polystyrene Colloidal Crystals Covalently Bound to a Capture Reagent

250 nm Carboxyl-Modified Polystyrene Microspheres (i.e., colloids) were purchased from Duke Scientific (Palo Alto, Calif.). The colloids were spun in a centrifuge at 9500 rpm for 15 minutes. Solvent was removed to bring the volume concentration of the colloids to ˜50% of the total volume of the solution. To prepare the array of colloidal crystals, freshly oxidized 18×18 glass coverslips were coated with n-octadecyltrichlorosilane (OTS) monolayers to generate a chemical template having lyophilic and lyophobic regions. Eight microliters of colloidal sol were physically confined between an OTS-coated coverslip and a freshly oxidized glass sealed with epoxy. The colloids were crystallized into colloidal crystals by incubation at 40° C. for at least 2 days, until they began to display photonic properties. The arrays of colloidal crystals were formed by physically separating the OTS-coated coverslip from the freshly oxidized glass.

A 200 nm spectral scan was performed on the arrays using a Cary 1e UV-Vis spectrophotometer and any array that did not exhibit a photonic stop band at the appropriate range of wavelengths (i.e. stop-band peak of ˜540 nm in air) was discarded.

To prepare arrays with protein conjugated to the colloidal crystals, the remaining arrays were placed in a petri dish with 3 ml of MES buffer (9.76 g of 2-Morpholinoethanesulfonic acid (MES)/ml H₂O) and 60 μl of EDC solution (12 mg of 1-Ethyl-3-[3-dimethylaminopropyl]carbodiimide HCl/ml MES). 100 μg of Goat anti-bovine antibody with or without a fluorescent label (Sigma, St. Louis, Mo.) was added immediately after the addition of EDC (Pierce Rockford, Ill.) and the arrays were incubated for at least one hour. The arrays were rinsed with phosphate buffered saline. Arrays comprising colloidal crystals conjugated to a fluorescently labeled Goat anti-bovine antibody were visually inspected using Nikon eclipse TE 2000-5 Fluoresence Microscope.

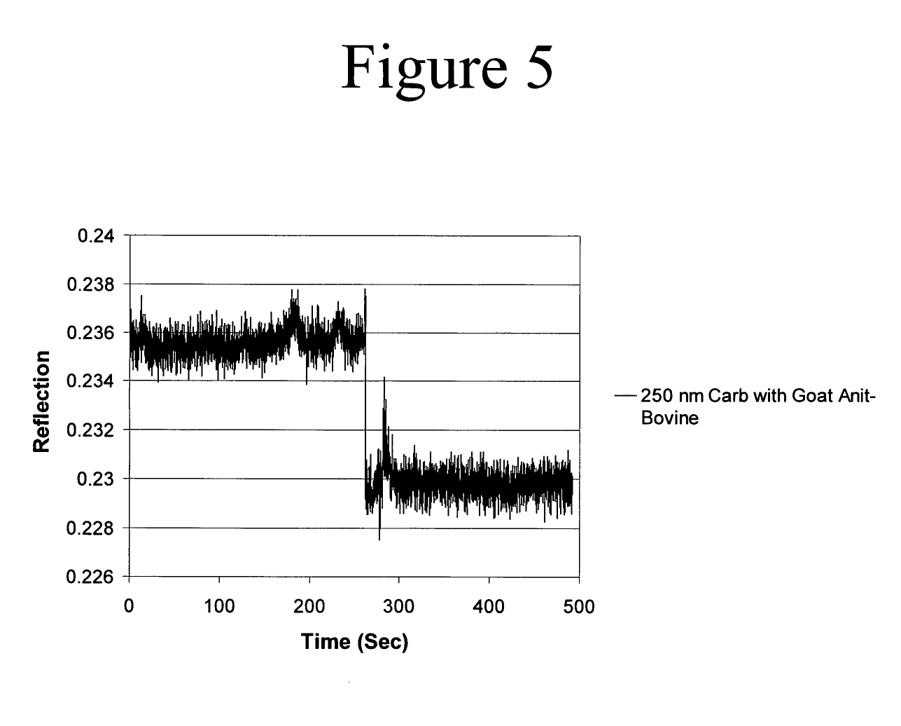

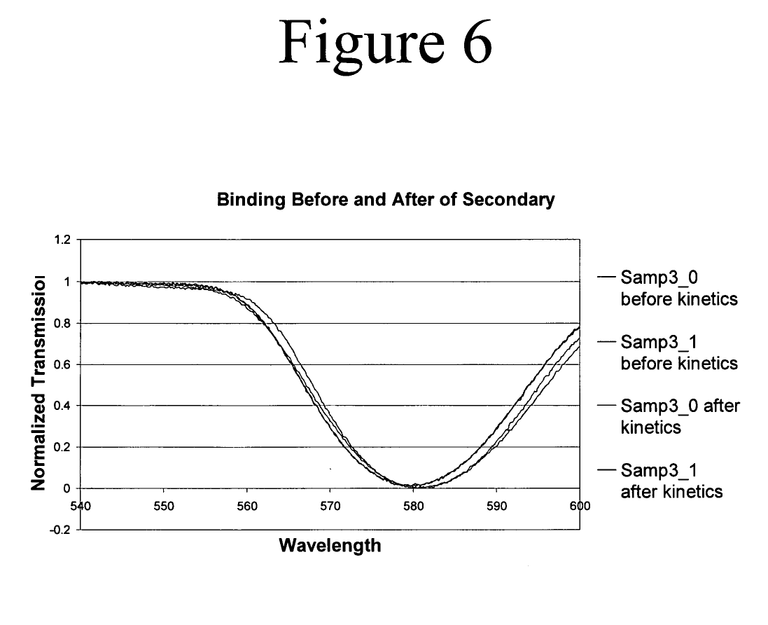

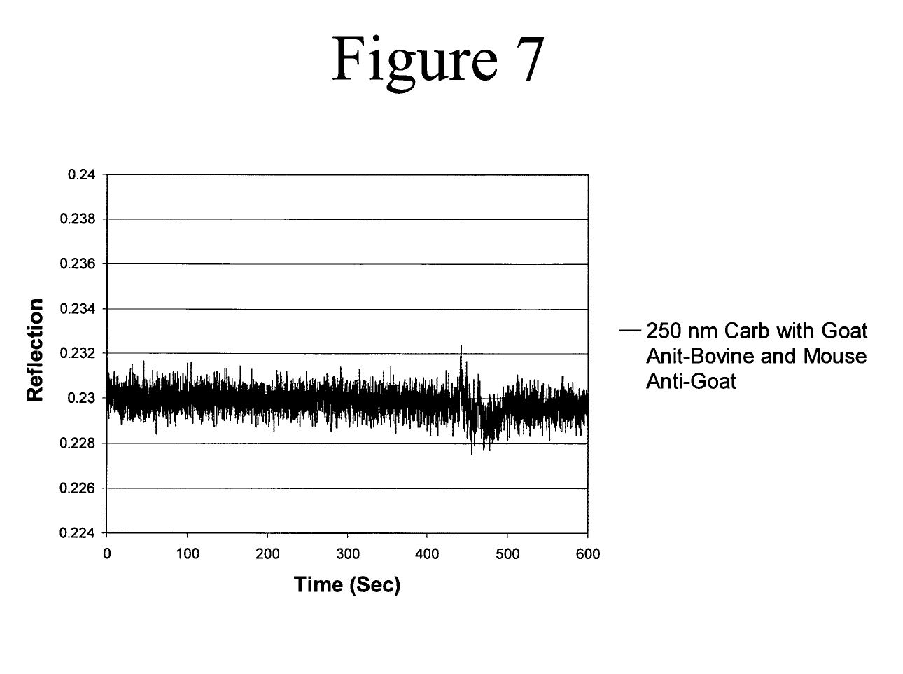

Arrays comprising colloidal crystals conjugated to an unlabeled goat anti-bovine antibody were placed in cuvettes and placed in the spectrophotometer. A continuous transmission intensity measurement was performed at 590 nm for ˜2 minutes to get a baseline reading. While continuing the scan, mouse anti-goat antibodies (Sigma, St Louis, Mo.) were introduced via a syringe pump. Almost immediately after introduction, a dramatic shift in intensity was observed (FIG. 5). In addition, several full spectrum scans were performed to identify the new peak (i.e., stop band) location following binding of the mouse anti-goat antibody to the goat anti-bovine antibody covalently attached to the crystal (FIG. 6). As a control, a buffer solution was injected into some cuvettes. No change in the stop band was detected. This data is depicted in FIG. 7.

### Example 6

### A Method for Assembling a Synthetic Lipid Bilayer using Colloidal Crystals Formation of Lipid Bilayers on Colloids prior to Crystallization

Lipids: 1-Palmitoyl-2-oleoyl-sn-glycero-3-phosphocholine(POPC), 1,2-dimyristoyl-sn-glycero-3-phosphocholine (DMPC), Dioleoyl-sn-Glycero-3-[Phospho-L-Serine] (DOPS) and 1-palmitoyl-2-[12-[(7-nitro-2-1,3-benzoxadiazol-4-1)amino]dode-canoyl]-sn-glycero-3-phospholcholine (NBDPC,16:0-12:0, tail-labeled) are purchased from Avanti Polar Lipids (Alabaster, Ala.). Additional lipids include Texas Red 1,2-dihexadecanoyl-sn-glycero-3-phosphoethanolamine (Texas Red-DHPE), purchased from Molecular Probes (Eugene, Oreg).

Preparing Lipids: Supported phospholipid bilayers are formed using a vesicle fusion and rupture method as described in, e.g., Tamm et al., Biophysical Journal 47(1): 105-113 (1985), and Yee et al., Adv. Mater. 16(14):1184-1189 (2004). Briefly, small unilamellar vesicles (SUVs) ware prepared using vesicle extrusion methods. Typically, a desired amount of lipid or lipid mixtures suspended in chloroform or chloroform/methanol mixtures is mixed in a glass vial. The solvent phase is then evaporated under a stream of nitrogen and subsequently evacuated for at least 1 h in a vacuum dessicator. The dried lipid mixture is then suspended in Millipore water and kept at 4° C. to be rehydrated overnight. The total lipid concentration is 2 mg/ml. The desired amount of hydrated aqueous solution is then sonicated and passed through a Avanti Mini-Extruder (Avanti, Alabaster, Ala.) using 0.1 μm polycarbonate membrane filters (Avanti, Alabaster, Ala.) for 21 times at a desired temperature (typically 10° C. above the transition temperature for the major lipids). One part of the resulting SUV solutions is diluted with one part of PBS and stored at 4° C. until use.

Fusing Lipids on individual colloids: Single lipid bilayer membranes are deposited on colloids by spontaneous fusion of SUVs such as discussed by M. M. Baksh, M. Jaros, and J. T. Groves, Nature, 2004, 427, 139 and Bayerl, T. M.; Bloom, M., Physical-Properties of Single Phospholipid-Bilayers Adsorbed to Micro Glass-Beads—a New Vesicular Model System Studied by H-2-Nuclear Magnetic-Resonance. Biophysical Journal 1990, 58, (2), 357-362. 25 μL of extruded lipids and 25 ρL of PBS are added to 100 μL of colloidal sol. This is then diluted with 850 μL of dH2O, and vortexed. Highly concentrated sol is then created using a Fischer A Microcentrifuge. The submicron silica colloids were spun for 5 minutes at 9500 rpm. The larger colloids were spun for 3 minutes at 5000 rpm. Solvent was removed to bring the concentration of colloids by volume to between 44% -56% . High concentration solutions were sonicated for 5 minutes then vortexed to resuspend the colloids.

Formation of Supported Lipid Bilayers on Pre-formed Colloidal crystals

Bilayer samples are prepared by placing a clean substrate surface over a ˜80 μl SUV drop placed at the bottom of a crystallization well. The sample is allowed to incubate for approximately 5 min to ensure equilibrium coverage. The well is then filled with buffer solution, transferred to a large reservoir of buffer in which the substrate is shaken gently to remove excess vesicles. The supported bilayer samples are stored in deionized water or PBS buffer.

### Example 7

### Formation of Continuous Fluid Lipid Bilayers on an Array of 330 nm Colloidal Silica Crystals

An array of 330 nm colloidal crystal was created as in Example 3. Single unilaminaer vesilces (SUV) were prepared as in Example 6. Surfaces of colloidal arrays are lightly oxidized by exposure to deep UV for 12 minutes as in Example 1. Colloidal samples were then dropped onto a ˜120 ul SUV drop placed at the bottom of a crystallization well. The sample is allowed to incubate for approximately 15 min to ensure highest coverage. The well is then filled with buffer solution, transferred to a large reservoir of buffer in which the substrate is shaken gently to remove excess vesicles. The supported bilayer samples are stored in deionized water or PBS buffer. Fluidity of the continuous fluid bilayer was confirmed by observing fluorescence recovery after photobleaching (FRAP) (see, e.g., Koppel et al., Biophys. J. 16:1315-1329 (1976) and Axelrod et al., Biophys. J. 6:1055-1069 (1976)).

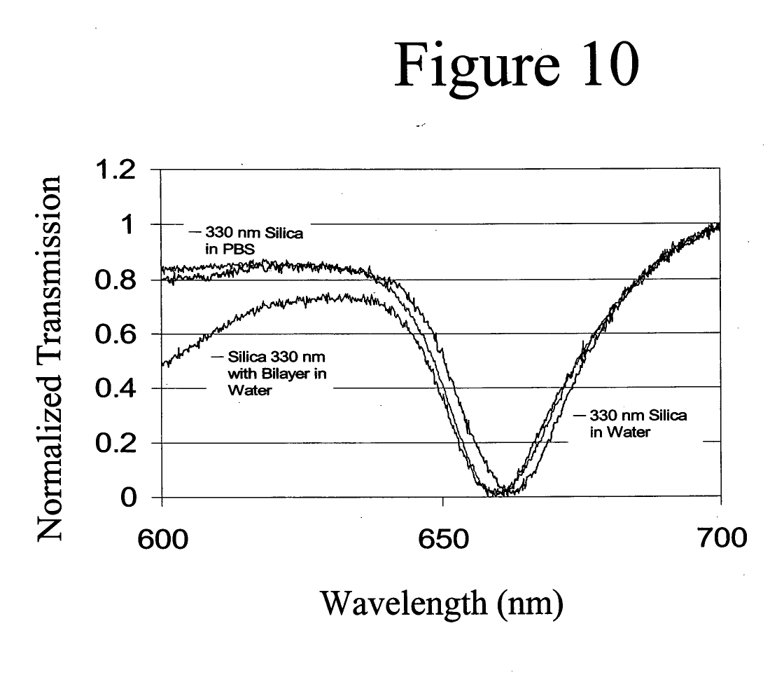

Several ˜200 nm spectral scans, before and after vesicle fusion, illustrate the photonic stop band, FIG. 10. Although the samples are rinsed in water, the PBS is retained in gaps between the colloids. The PBS is represented in the shifted spectrum of the bilayer colloidal crystal in water.

Although the foregoing invention has been described in some detail by way of illustration and example for purposes of clarity of understanding, one of skill in the art will appreciate that certain changes and modifications may be practiced within the scope of the appended claims. In addition, each patent, patent publication, and reference provided herein is incorporated by reference in its entirety to the same extent as if each patent, patent publication, and reference was individually incorporated by reference.

1. An array of colloidal crystals on a solid support, said array comprising colloidal crystals having uniform structural and photonic properties, wherein the colloidal crystals are spaced apart from each other.

2. The array of claim 1, wherein the colloidal crystals are at least about 500 nm in size.

3. The array of claim 1, wherein the array has a pitch of about 1:1.

4. The array of claim 1, wherein the colloids are selected from the group consisting of: polymeric colloids, inorganic colloids, metal colloids, ceramic colloids, coated colloids, semiconductor colloids, and combinations thereof.

5. The array of claim 4, wherein said colloids are polystyrene colloids.

6. The array of claim 4, wherein said colloids are silica colloids.

7. The array of claim 1, wherein the colloidal crystals comprise colloids from about 100 nm to about 10 μm in size.

8. The array of claim 1, wherein the colloidal crystals comprise functionalized colloids.

9. The array of claim 8, wherein the colloids are functionalized with a functional group selected from the group consisting of: a carboxyl, an amino, an amido, an amidino, and combinations thereof.

10. The array of claim 8, wherein the colloids are functionalized with a lipid bilayer.

11. The array of claim 8, wherein the colloidal crystals further comprise a capture reagent.

12. A method of preparing said array of claim 1, said method comprising the steps of:
(a) contacting said colloids with a chemical template having lyophilic and lyophobic regions;
(b) crystallizing said colloids into said colloidal crystals; and
(c) removing said chemical template to prepare said array of colloidal crystals.

13. The method of claim 12, wherein said method further comprises the step of physically confining said colloids prior to step (a).

14. The method of claim 12, wherein said colloids are at a concentration of about 20% to about 75% by volume prior to the contacting step.

15. The method of claim 14, wherein said colloids are at a concentration of about 44% to about 56% by volume.

16. The method of claim 12, wherein the colloids are selected from the group consisting of: polymeric colloids, inorganic colloids, metal colloids, ceramic colloids, coated colloids, semiconductor colloids, and combinations thereof.

17. The method of claim 16, wherein said colloids are polystyrene colloids.

18. The method of claim 16, wherein said colloids are silica colloids.

19. The method of claim 12, wherein the colloidal crystals comprise colloids from about 100 nm to about 10 μm in size.

20. The method of claim 12, wherein the colloidal crystals comprise colloids that are functionalized.

21. The method of claim 20, wherein the colloids are functionalized with a functional group selected from the group consisting of a carboxyl, an amino, an amido and an amidino.

22. The method of claim 20, wherein the colloidal crystals further comprise a capture reagent.

23. The method of claim 22, wherein the capture reagent is selected from the group consisting of: a receptor, a ligand, an antibody, a nucleic acid, a polysaccharide, and combinations thereof.

24. The method of claim 22, wherein the capture reagent is an antibody.

25. The method of claim 20, wherein the colloids are functionalized with a lipid bilayer.

26. The method of claim 25, wherein each of said colloidal crystals of said array are functionalized with a lipid bilayer.

27. A method for detecting an analyte in a sample, said method comprising:
(a) contacting a sample suspected of containing the analyte with an array of colloidal crystals comprising colloidal crystals having uniform structural and photonic properties; and
(b) detecting binding of the analyte to the colloidal crystals.

28. The method of claim 27, wherein said sample is a biological sample.

29. The method of claim 27, wherein the analyte is selected from the group consisting of: a polypeptide, a nucleic acid, a lipid, a polysaccharide, a bacteria, a virus, a trace-metal, and combinations thereof.

30. The method of claim 27, wherein the colloidal crystals comprise functionalized colloids.

31. The method of claim 27, wherein said detecting comprises measuring a change in a stop band property of the colloidal crystals.

32. The method of claim 31, wherein the stop band property is selected from the group consisting of: an intensity shift, a wavelength shift, a width shift, and combinations thereof.

33. The method of claim 27, wherein said detecting comprises spectroscopy.

34. An apparatus comprising:
an array of claim 1;
a radiation source for directing radiation to the colloidal crystals; and
a detector adapted to detect radiation from the colloidal crystals.

35. The apparatus of claim 34, wherein the radiation source is a light source.
